(19) United States
(12) Patent Application Publication
(10) Pub. No.: US 2003/0167022 A1
(43) Pub. Date: Sep. 4, 2003

(54) Catheter for measuring pressure

(76) Inventor: Gerrat Dijkman, Enschede (NL)

Correspondence Address: Robert A Jensen, Suite 1020, 2033 6th Avenue, Seattle, WA, 98121, US

(21) Appl. No.: 10/343,159

(22) PCT Filed: Jul. 25, 2001
(86) PCT No.: PCT/NL01/00575

(51) Int. Cl. A61B 5/00

(52) U.S. Cl. 600/561

(57) ABSTRACT

The invention relates to a catheter for performing an in vivo pressure measurement, comprising a catheter tube with at least one channel for a pressure-conducting, medium, a pressure sensor (4,5) for placing in contact with the medium and a liquid space (40) provided with at least two ports (41,43) where in the at least one channel is connected to a first port (41), characterized in that the liquid space is a pressure chamber (40) in which the pressure sensor (4,5) is accommodated. The invention also relates to a pressure chamber as described as component of a catheter according to the invention. The invention likewise relates to a connector unit comprising a housing provided with one or more recesses which are each intended to receive a pressure chamber (40) of a catheter according to the invention, wherein electronic means for connecting to the pressure sensor (4,5) are arranged in the housing.

The invention relates to a catheter for performing an in vivo pressure measurement, comprising a catheter tube with at least one channel for a pressure-conducting medium, a pressure sensor for placing in contact with the medium and a liquid space provided with at least two ports, wherein the at least one channel is connected to a first port.

A catheter is known wherein the pressure sensor is arranged in a measuring arrangement and is in contact via a pressure-conducting membrane with liquid in a liquid conduit connected to the third port of a three-way tap provided with four ports, wherein the first port can optionally also be connected to the fourth port which is connected to a flushing line.

During a pressure measurement with the known catheter the catheter tube is flushed and filled with water, wherein the first and fourth port are interconnected. The catheter tube is then introduced into the patient at the intended position, for instance the urinary bladder, and water is introduced into the bladder via a channel (“lumen”) present for this purpose in the catheter tube. The signal of the pressure sensor is subsequently set to zero using a compensation signal in a situation where the pressure on the sensor equals the ambient pressure. The third and the second port are interconnected for this purpose. After this so-called balancing of the sensor signal, the first and the third port are interconnected and the pressure in the patient, in this case in his/her bladder, can be measured.

The known catheter has the advantage that it has a low purchase price and can be supplied in, sterile state.

The use of the known catheter does have drawbacks. The placing and venting of the catheter takes a relatively long time due to the inherent use of many components, such as a pressure tube, a tap operating as three-way tap and having four ports and, in the case a disposable pressure tube is used, a pressure-conducting medium, for instance a so-called “dome”, in order to separate the pressure sensor to be reused from the part of the catheter to be discarded after use. Another significant drawback is inherently related to the applied measurement technique; because the pressure is measured at a fixed position relatively far outside the patient at the same height as the patient, the measurement result greatly depends upon the degree to which the patient can remain in the same position between balancing of the pressure sensor and the actual measurement.

It is an object of the invention to provide a catheter which, while retaining the advantages of the known catheter, does not have its drawbacks.

This objective is achieved, and other advantages gained, with a catheter of the above described type, wherein according to the invention the liquid space is a pressure chamber in which the pressure sensor is accommodated.

In a catheter according to the invention the pressure sensor is accommodated in a pressure chamber which is used to balance the sensor, which tap is placed directly on the end of the catheter tube and is thus situated at the same height as the catheter tube. Consequently the measurement result does not depend on the extent to which a patient can remain in the same position, since a movement of the patient cannot result in a difference in height of sensor and catheter tube.

Additional advantages of a catheter according to the invention are the reduced number of components, resulting in a reduced environmental impact, and the extremely short time required to place the catheter.

In a first preferred embodiment the pressure chamber is provided with a tap provided with three ports, wherein the at least one channel is connected to a first port, and a second port is in open communication with the environment, and the tap is adapted for connection of the third port as required to the first or the second port, and wherein the pressure sensor is provided in the third port in close proximity to the first port. By means of this embodiment the catheter can be balanced in a very short time, even after being introduced into the patient.

In a second embodiment of a catheter according to the invention, wherein the catheter tube comprises at least two channels, wherein each channel is connected to the first port of a pressure chamber provided with at least two ports and wherein a pressure sensor is provided in each pressure chamber.

Using such a catheter it is possible to perform pressure measurements at a number of locations simultaneously in the relevant organ of a patient.

Depending on the embodiment and the number of lumina, a catheter according to the invention is for instance suitable for measuring pressure in bladder, urethra or rectum (for which purpose the catheter tube is provided with a balloon for filling with water). The catheter is likewise suitable for invasive measurement of blood pressure or for measuring cranial pressure.

The invention also relates to a pressure chamber as described as component of a catheter according to the invention.

The invention likewise relates to a connector unit comprising a housing provided with one or more recesses which are each intended to receive a pressure chamber of a catheter according to the invention, wherein electronic means for connecting to the pressure sensor are arranged in the housing.

The invention will now be elucidated hereinbelow on the basis of embodiments and with reference to the drawings.

In the drawings

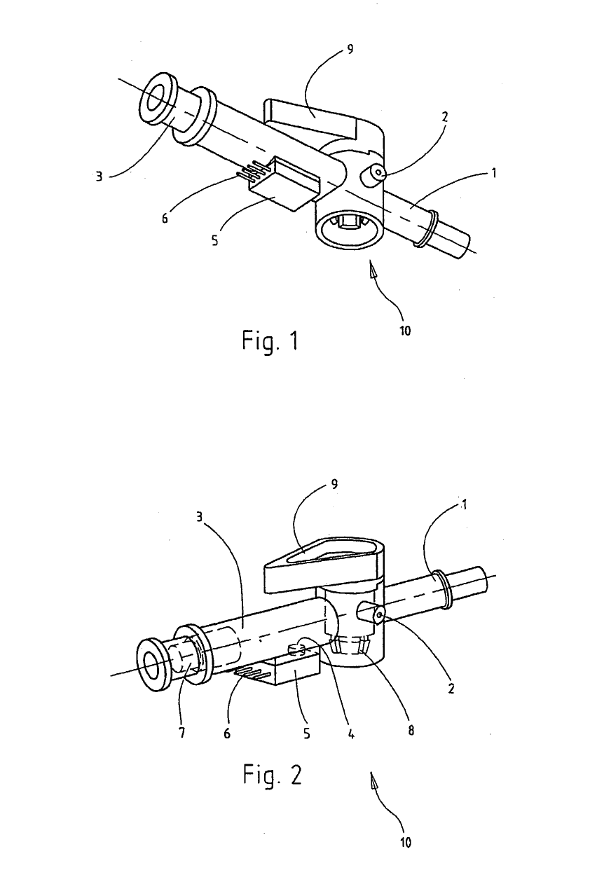

FIG. 1 shows a perspective bottom view of a two-way tap provided with a pressure sensor for a catheter according to the invention,

FIG. 2 shows in cut-away perspective view the two-way tap depicted in FIG. 1,

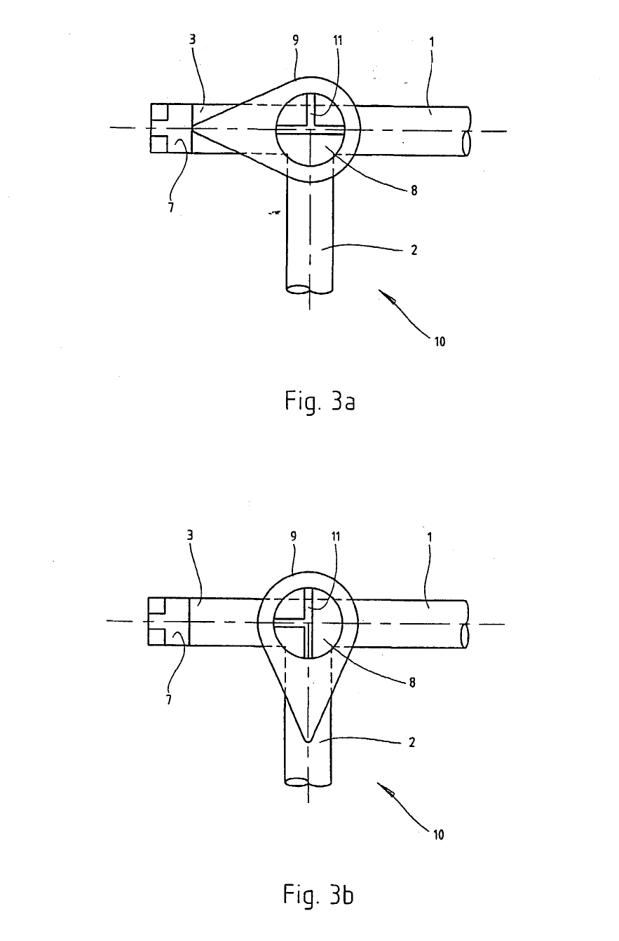

FIGS. 3a, 3b show schematic views of the positions of the tap depicted in FIG. 1,

FIG. 4 shows a first embodiment of a catheter tube connectable to a two-way tap according to the invention,

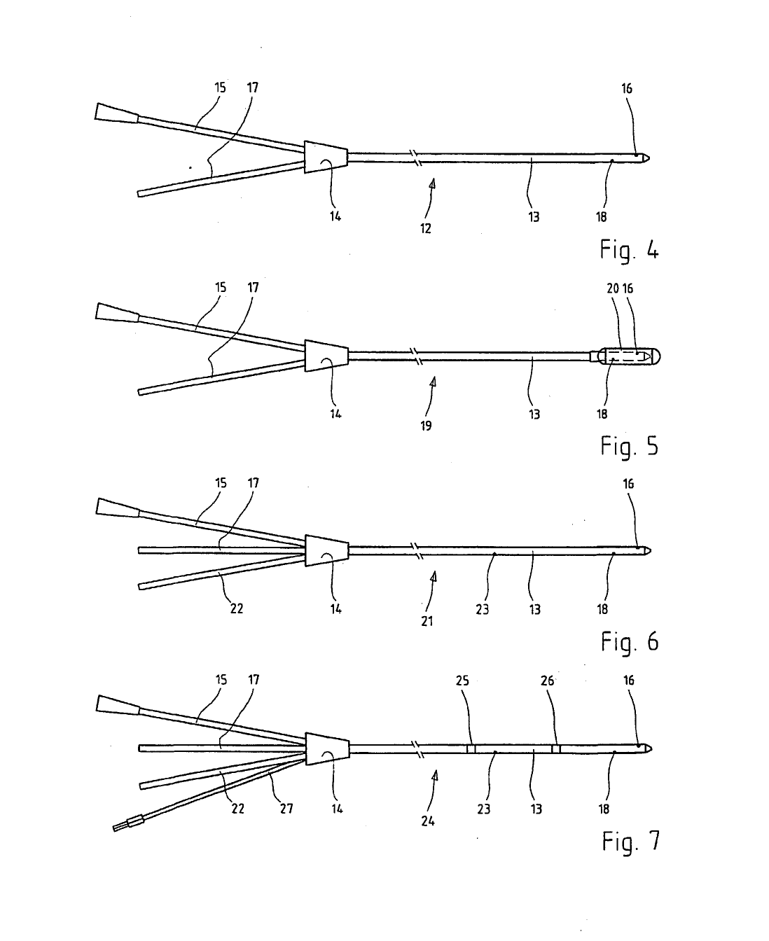

FIG. 5 shows a second embodiment of a catheter tube connectable to a two-way tap according to the invention,

FIG. 6 shows a third embodiment of a catheter tube connectable to a two-way tap according to the invention,

FIG. 7 shows a fourth embodiment of a catheter tube connectable to a two-way tap according to the invention,

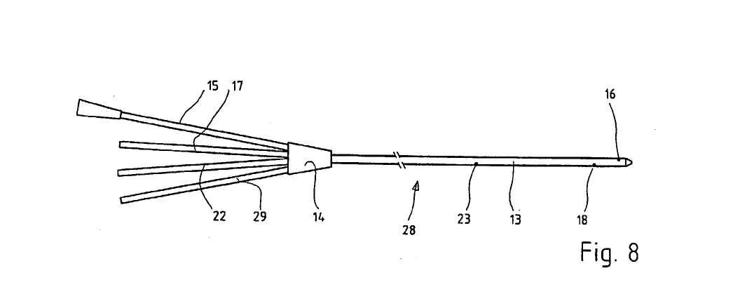

FIG. 8 shows a fifth embodiment of a catheter tube as component of a catheter according to the invention, and

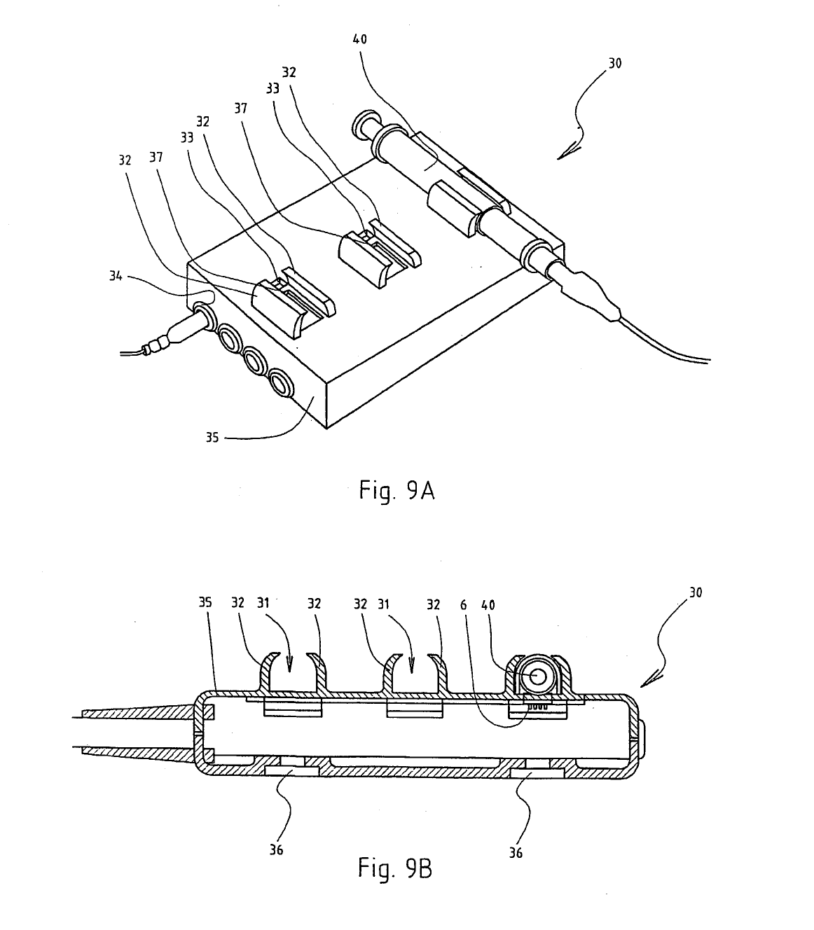

FIG. 9A shows in schematic perspective, view a connector unit for reading the pressure sensor(s), and

FIG. 9B shows a schematic cross-section of the connector unit of FIG. 9A,

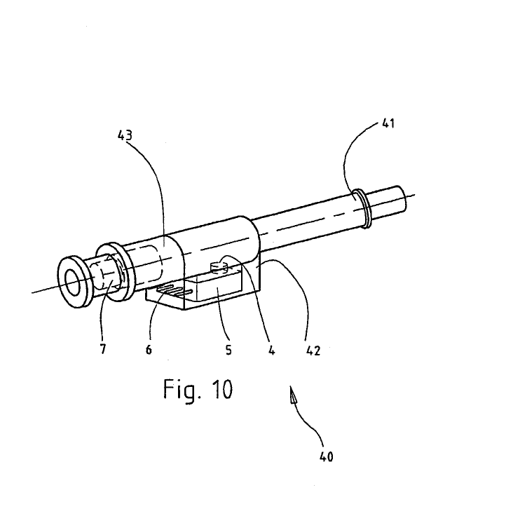

FIG. 10 shows a pressure chamber provided with an integrated pressure spoor for a catheter according to the invention.

Corresponding components are designated in the figures with the same reference numerals.

FIGS. 1 and 2 show a two-way tap 10 provided with three ports 1, 2, 3, wherein a lumen of a catheter tube (not shown) can be connected to the first port 1, and the second port 2 is in open communication with the environment. Situated in the third port 3 in close proximity to the first port is the pressure-sensitive surface 4 of a pressure sensor 5 which is provided with connecting pins 6. The third port 3 is closed by means of a non-return valve 7. The figure further shows the plug 8, which is provided with bores in T-shape, and the arrow-shaped handle 9 of tap 10.

FIG. 3a shows the two-way tap 10 in schematic top view at the start of a pressure measurement, wherein the catheter tube (not shown) connected to the first port 1 and not yet introduced into the patient is flushed with liquid injected into the first channel using a syringe via non-return valve 7 and T-shaped channel 11 in plug 8. After flushing, the handle 9 is rotated a quarter-turn to the left to the position shown in FIG. 3b.

The use of a non-return valve eliminates the need for an additional tap for the purpose of flushing and venting the catheter.

FIG. 3b shows two-way tap 10 in the situation prior to balancing of the pressure sensor (not shown) arranged in the third port 3, wherein third port 3 is in open communication with the environment via T-shaped channel 11 and second channel 2. After balancing, the handle 9 is rotated a quarter-turn to the right back into the situation shown in FIG. 3a, wherein third port 3 and the pressure sensor placed therein is in open communication with the first port 1 and the catheter lumen connected thereto.

FIG. 4 shows a catheter tube 12 for measuring pressure in a part of a patient which is naturally filled with liquid, for instance a bladder or urethra, with a part 13 for introducing into a patient which is provided with a manifold 14, a first lumen 15 which debouches at the end of part 13 into an opening 16 for introducing liquid into the patient, and a measuring lumen 17 which can be coupled to the third port 3 of two-way tap 10 shown in FIGS. 1-3b. Via an opening 18 in tube part 13 the measuring lumen 17 is in open communication with the liquid introduced into the patient via opening 16.

FIG. 5 shows a catheter tube 19 for measuring the pressure in a part of a patient which naturally contains no water, for instance the rectum. The end of tube part 13 with openings 16, 18 is enclosed by a balloon of polyurethane with a diameter of about 1 cm, which is filled with liquid via the first lumen 15 and opening 16, the pressure of which is measured via opening 18 and the liquid in lumen 17.

FIG. 6 shows a catheter tube 21 provided with a second measuring lumen 22 which via a second measuring opening 23 in tube part 13 is in open communication at a second position with the liquid introduced into the patient via opening 16. The second measuring lumen 22 can be coupled to the third port 3 of a second two-way tap 10.

FIG. 7 shows a catheter tube 24 which corresponds with the catheter tube 21 of FIG. 6 but which is however augmented with two electrodes 25, 26 round tube part 13, provided with a connecting cable 27,to measure the electrical resistance of the section between electrodes 25, 26 so as to enable detection of possible leakage of liquid, for instance urine into the urethra.

FIG. 8 shows a catheter tube 28 which compared to the catheter tube shown in FIG. 6 is augmented with a third measuring lumen 29 which via a third measuring opening (not shown) in tube part 13 is in open communication at a third position with the liquid introduced into the patient via opening 16. The third measuring lumen 29 can be coupled to the third port 3 of a third two-way tap 10. The number of measuring lumina with pressure sensors for reading can of course be further increased or reduced as desired.

FIG. 9A shows a connector unit 30 with a housing 35 which is provided with a number of recesses 31, each of which is intended to receive a port 3, 43 of a pressure chamber 10, 40 of a catheter according to the invention (see FIGS. 1, 2 and 10). An opening 33 for pressure sensor 4,5 is arranged in each recess. Recesses 31 are further provided with standing edges 32 for fixing the ports of the pressure chambers in the correct position. The deeper levels 37 are arranged for reception of the pressure sensors, possibly provided with a protecting housing, such as housing 42 in FIG. 10.

FIG. 9B shows connector unit 30 in cross-section. Here can be seen that pressure sensor 4,5 is provided with pins 6 which protrude inward into the housing to enable placing thereof in contact with further electronic means (not shown) for reading the measurement values of the pressure sensor. Connector unit 30 is provided on the side with electrical connections 34. Diverse suitable electronic means for reading such commercially available pressure sensors are likewise commercially obtainable and are therefore known to a skilled person in this technical field. The connector unit can for instance comprise connecting cables for connection of the pressure sensors to a separate data-processing unit. Alternatively, a pre-amplifier can be arranged in the connector unit for a first processing of the data from the pressure sensors. The processed data is then transmitted via connecting cables to a separate data-processing unit.

Connector unit 30 can be attached to a patient by means of a number of surface electrodes and is suitable for both stationary and ambulatory use. For this purpose openings 36 are arranged in housing 35 for receiving the usual fastening means present on known surface electrodes. An earth connection is preferably arranged on the fastening means. In the shown preferred embodiment the connector unit 30 is suitable for simultaneous reading of three pressure sensors. The three pressure sensors for reading can provide measurement values of for instance the bladder pressure (twice) and the urethra pressure, which can be measured simultaneously using a catheter according to the invention. FIG. 8 shows a preferred embodiment of a catheter according to the invention which is suitable for this purpose.

FIG. 10 shows a pressure chamber 40 which is provided with two ports 41 and 43. Port 41 is intended for connection to a lumen of a catheter tube. Port 43 is provided with a non-return valve 7. A pressure sensor 4,5 is received in pressure chamber 40. Pressure chamber 40 is partially provided with a housing 42 to protect the pins 6 of pressure sensor 4,5. If desired, such a protecting housing can also be applied to pressure chamber 10 in FIGS. 1 and 2.

Pressure chamber 40 is a simpler embodiment of the invention. The foregoing figures show the preferred embodiment with two-way tap. This provides the additional option of connecting the first port to the environment so as to still enable balancing of the catheter even after it has been introduced into the patient. This is an option and not essential as such to the invention. It is therefore noted with emphasis that everywhere in the preceding description “(two-way) tap” can be understood to mean “pressure chamber”.

In summary, the invention teaches the connection of a pressure chamber to the lumen of a catheter tube and the integration of a pressure sensor in the pressure chamber. The pressure chamber can optionally be provided with a two-way tap. The pressure chamber and the lumen are preferably connected fixedly to each other, for instance by means of glueing.

In a catheter according to the invention the number of lumina with pressure chamber and sensor integrated therein can be chosen as desired, so that multiple measurements can be performed simultaneously. The catheter according to the invention is a disposable article and is highly suitable for medical applications in general and urological applications in particular.

It is noted that the shown embodiments are in no way intended to limit the scope of the invention, but serve only for purposes of elucidation. The invention is not therefore limited to the shown and described embodiments, but extends generally to any embodiment which falls within the scope of the appended claims as seen in the light of the foregoing description and drawings. This particularly includes all combinations of the shown and discussed embodiments which can be realized by a skilled person. While the invention is outlined in the context of urological applications, it is also particularly suitable for many other medical applications, such as in the field of gastro-enterology or for measuring cranial pressure, for instance after an invasive treatment.

1. Catheter for performing an in vivo pressure measurement, comprising a catheter tube (12, 19, 21, 24, 28) with at least one channel (17) for a pressure-conducting medium, a pressure sensor (4, 5) for placing in contact with the medium, and a liquid space (10, 40) provided with at least two ports (1, 3, 41, 43), wherein the at least one channel (17) is connected to a first port (1), characterized in that the liquid space is a pressure chamber (1) in which the pressure sensor (4, 5) is accommodated.

2. Catheter as claimed in claim 1, wherein the pressure chamber is provided with a tap (10) provided with three ports (1, 2, 3), wherein the at least one channel (17) is connected to a first port (1) and a second port (2) is in open communication with the environment, and the tap (10) is adapted for connection of the third port (3) as required to the first (1) or the second port (2), and wherein the pressure sensor (4, 5) is provided in the third port (3) in close proximity to the first port (1).

3. Catheter as claimed in any of the foregoing claims, wherein the catheter tube (19, 21, 23) comprises at least two channels (17, 22), wherein each channel (17, 22) is connected to the first port (1) of a pressure chamber (10, 40) provided with at least two ports, and wherein a pressure sensor (4, 5) is provided in each pressure chamber (10, 40).

4. Pressure chamber as described as component of a catheter as claimed in any of the foregoing claims.

5. Connector unit comprising a housing (30) provided with one or more recesses (31) which are each intended to receive a pressure chamber (10, 40) of a catheter as claimed in any of the foregoing claims, wherein electronic means for connecting to the pressure sensor (4, 5) are arranged in the housing.
